(19) United States
(12) Patent Application Publication
(10) Pub. No.: US 2010/0015645 A1
(43) Pub. Date: Jan. 21, 2010

(54) IL-8 AS BIOMARKER FOR THE DETECTION OF UROLITHIASIS

(75) Inventors: Suh-Hang Hank Juo, Kaohsiung (TW); Jau-Ling Suen, Kaohsiung (TW); Yii-Her Chou, Kaohsiung (TW)

Correspondence Address: LANDO & ANASTASI, LLP, ONE MAIN STREET, SUITE 1100, CAMBRIDGE, MA, 02142, US

(73) Assignee: KAOHSIUNG MEDICAL UNIVERSITY, Kaohsiung (TW)

(21) Appl. No.: 12/175,674

(22) Filed: Jul. 18, 2008

(51) Int. Cl. G01N 33/53; G01N 33/00; G01N 33/543

(52) U.S. Cl. 435/7.92; 436/86; 436/518

(57) ABSTRACT

Disclosed herein is a method for the detection or preliminary screening of urolithiasis, comprising: detecting the IL-8 level and the creatinine level in a urine sample taken from a human subject suspected to have urolithiasis; obtaining a creatinine-normalized IL-8 level in the urine sample by normalizing the detected IL-8 level to the detected creatinine level; and comparing the creatinine-normalized IL-8 level in the urine sample with a predetermined standard; wherein an elevation of the creatinine-normalized IL-8 level in the urine sample as compared to the predetermined standard is indicative of urolithiasis.

### BACKGROUND OF THE INVENTION

1. Field of the Invention

This invention relates to the finding of interleukin-8 (IL-8) as a biomarker for the detection of urolithiasis. Based on this finding, the invention provides a method for detecting, preliminary screening or monitoring urolithiasis, in which IL-8 is used as a biomarker for urolithiasis,

2. Description of the Related Art

Urolithiasis, a condition involving the development of stones in the kidney, bladder, and/or urinary tract, is a common disease with a prevalence of 5-10% worldwide. It is a rapidly increasing universal problem with an important influence on the health care industry. Urolithiasis is a multifactorial disease, and its underlying etiology is not yet well understood. The risk factors for developing urinary stones include genetics, age, sex, geography, seasonal factors, diet and occupation (M. Monga et al. (2006), J. Urol., 175(6): 2125-2128).

Urine is normally supersaturated with oxalate ions and calcium. Under suitable conditions, it will lead to the formation of calcium oxalate crystals. These crystals can be retained in the kidneys via binding to the tubular cells (Dirk J. Kok et al. (1994), Kidney International, 46:847-854), and then aggregate to form large ones. Thus, crystal growth, aggregation and retention are all important aspects of the development of urolithiasis.

During certain hyperoxaluric conditions, retained crystals move into the interstitium to induce non-infectious inflammation. Several studies have shown that renal stones could stimulate renal cells to secrete inflammatory mediators in vitro, such as monocyte chemoattractant protein-1 (MCP-1, also known as CCL2) (Tohru Umekawa et al. (2002), Kidney International, 61:105-112) and tumor necrosis factor-alpha (TNF-α)(R. de Water et al. (2001), Am. J. Kidney Dis., 38(2):331-8). Thus, the inflammatory responses of renal tissue play an important role in the disease process of urolithiasis (Saeed R. Khan (2004), Clin. Exp. Nephrol., 8(2):75-88).

Currently, most patients with urolithiasis are diagnosed after the development of symptoms. A reliable biomarker for urolithiasis could lead to earlier diagnosis, treatment, and better monitoring of the disease course. However, very few studies have examined in detail the urinary inflammatory cytokines and chemokines in patients with urolithiasis.

In J. Urol., December 1998, 160: 2284-2288, Eugene Rhee et al. evaluated the possible role of cytokines IL-1β, IL-1α and IL-6 in patients with urolithiasis. They compared the urine samples of patients with urolithiasis, patients with bacterial cystitis and normal subjects and found that patients with urolithiasis showed significant elevations in IL-6 without marked increases in either IL-1β or IL-1α relative to normal subjects, whereas patients with bacterial cystitis showed significant elevations in IL-6, IL-1β and IL-1α. The results of Eugene Rhee et al. reveal that IL-6 alone cannot distinguish bacterial cystitis from urolithiasis, but the combination of IL-β and IL-6 may do so.

In view of the aforesaid, it is highly desirable to explore a predictive biomarker for urolithiasis. The applicants then investigated whether the urine inflammatory cytokine and chemokine profiles from patients with urolithiasis could be used as diagnostic markers for urolithiasis. The applicants found from experiments that urinary IL-8 may be useful as a potential biomarker for the detection of urolithiasis.

### SUMMARY OF THE INVENTION

Therefore, according to a first aspect, this invention provides a method for the detection or preliminary screening of urolithiasis in a human subject, comprising:
detecting the IL-8 level and the creatinine level in a urine sample taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
   wherein an elevation of the creatinine-normalized IL-8 level in the urine sample as compared to the predetermined standard is indicative of urolithiasis.

In a second aspect, this invention provides a method for monitoring urolithiasis in a human subject, comprising:
detecting the IL-8 level and the creatinine level in a urine sample periodically taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
   wherein an elevation of the creatinine-normalized IL-8 level in the urine sample as compared to the predetermined standard is indicative of urolithiasis.

### BRIEF DESCRIPTION OF THE DRAWINGS

Other features and advantages of the present invention will become apparent in the following detailed description of the preferred embodiment with reference to the accompanying drawings, of which:

FIG. 1 shows the representative data of chemokine levels as detected by cytometric bead arrays from healthy controls (panel A) and patients with urolithiasis (panel B), in which dot plots yellow-A (585 nm) versus red-A (650 nm) representing different chemokines (IL-8, RANTES, Mig, MCP-1 and IP-10) are indicated in the figure by five discrete microparticle dye intensities along the y-axis; and the x-axis, which shows the values for sample fluorescence intensities, reflects the concentrations of the various biomarkers; and

FIG. 2 shows a comparison of urinary chemokine and cytokine concentrations in patients with urolithiasis (Δ) and normal subjects (◯), in which spots represent the values of creatinine-adjusted chemokine/cytokine concentrations, and the horizontal dashed lines indicate the normal range cutoff of each chemokine or cytokine, expressed as mean plus 2 SDs of the control group (creatinine-adjusted cutoff values, 25.83 pg/mg creatinine for IL-8, 10.81 pg/mg creatinine for RANTES, 475.63 pg/mg creatinine for Mig, 745.27 pg/mg creatinine for IP-10, and 6.11 pg/mg creatinine for IL-6). The difference between patients and controls was assessed by the chi-square test.

### DETAILED DESCRIPTION OF THE INVENTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described. For clarity, the following definitions are used herein.

The prevalence of urolithiasis in the general population has been increasing recently. Excess renal crystals stimulate an army of responses inducing local injury and non-infectious inflammation in the kidneys. These inflammatory responses may play an important role in the development of urolithiasis.

During inflammation, cytokines and chemokines play an important role in linking innate and adaptive immunity. Tissue macrophages or dendritic cells stimulated by toll-like receptor (TLR) can secrete various chemokines, such as interleukin 8 (IL-8, also known as CXCL8), RANTES (regulated on activation, normal T-cell expressed and secreted, also known as CCL5), macrophage inflammatory protein-1alpha (MIP-1 α, also known as CCL3), and MIP-1β (also known as CCL4)(Fabio Re and Jack L. Strominger (2001), J. Biol. Chem., 276(40):37692-37699).

IL-8 is the key chemoattractant for neutrophils, while RANTES, MIP-1α and MIP-1β are chemotactic factors for immature dendritic and natural killer (NK) cells. NK cells are an important source of interferon-gamma (IFN-γ), which induces the production of monokine induced by IFN-γ (Mig, also known as CXCL9) and IFN-γ inducible 10-kd protein (IP-10, also known as CXCL10). In turn, Mig and IP-10 attract activated T cells to inflamed sites (Thais P. Salazar-Mather et a. (2000), J. Clin. Invest., 105(7): 985-993).

Furthermore, macrophages and endothelial cells can produce MCP-1/CCL2 to attract additional macrophages (Kouji Matsushima et al. (1989), J. Exp. Med., 169:1485-1490; Teizo Yoshimura), (1989), J. Exp. Med., 169:1449-1450), while immature dendritic cells stimulated by pathogens can produce IL-12, TNF-α or IL-10 (Jacques Banchereau & Ralph M. Steinman (1998), Nature, 392(19): 245-252).

To examine these secreted inflammatory mediators in the context of urolithiasis, the applicants simultaneously compared the concentrations of five inflammatory cytokines and five inflammatory chemokines in the midstream morning urine specimens between patients with urolithiasis and healthy controls via multiplex immunoassays. It was surprisingly found that after creatinine normalization, urinary levels of IL-8, RANTES, MCP-1, IP-10, Mig and IL-6 were significantly increased in patients as compared to healthy controls. However, concentrations of urinary IL-1β, IL-10, IL-12, and tumor TNF-α were not significantly different between patients with urolithiasis and healthy controls. Using receiver operating characteristics (ROC) curve analysis, the applicants found that the cutoff point for the creatinine-normalized level of IL-8 was 6.2 pg/mg creatinine. With this value, the diagnostic sensitivity and specificity could reach 91% and 68%, respectively.

Accordingly, this invention provides a method for the detection or preliminary screening of urolithiasis in a human subject, comprising:
detecting the IL-8 level and the creatinine level in a urine sample taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
   wherein an elevation of the creatinine-normalized IL-8 level in the urine sample as compared to the predetermined standard is indicative of urolithiasis.

This invention also provides a method for monitoring urolithiasis in a human subject, comprising:
detecting the IL-8 level and the creatinine level in a urine sample periodically taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
   wherein an elevation of the creatinine-normalized IL-8 level in the urine sample as compared to the predetermined standard is indicative of urolithiasis.

Creatinine is a byproduct of muscle activity and cleared by the kidneys and excreted in urine (M. F. Boeniger et al. (1993), Am. Ind. Hyg. Assoc. J., 54(10):615-27) Creatinine is made at a steady rate and is not affected by diet or by normal physical activities. Its concentration in urine is a reliable reference value to standardize urinary solute excretion in a spot urine sample. Therefore, in the invention, creatinine was used to correct concentrations of cytokines/chemokines in urine.

According to this invention, the urine sample may be taken from a human subject at any time. Preferably, the urine sample is a first morning urine sample of the human subject, and more preferably a midstream sample of the first morning urine.

The IL-8 level may be measured by any means known to those skilled in the art. According to this invention, it is generally preferred to detect the quantity or level of IL-8 in the urine sample by immunoassays, including, but not limited to: multiplex immunoassay, enzyme linked immunosorbent assay (ELISA), radioimmunoassay (RIA), immunoradiometric assay (IRMA), etc.

According to this invention, quantifying IL-8 is conducted using an antibody-based binding moiety that specifically binds IL-8. In a more preferred embodiment of this invention, the antibody-based binding moiety is labeled with a detectable label selected from the group consisting of a radioactive label, a hapten label, a fluorescent label, and an enzymatic label.

The term “antibody-based binding moiety” or “antibody” includes immunoglobulin molecules and immunologically active determinants of immunoglobulin molecules, e.g., molecules that contain an antigen-binding site that specifically binds (immunoreacts with) to IL-8. The term “antibody-based binding moiety” is intended to include whole antibodies of any isotype (e.g., IgG, IgA, IgM, IgE, etc.), and includes fragments thereof that are also specifically reactive with IL-8.

According to this invention, the term “antibody-based binding moiety” or “antibody” includes a capture antibody and a detection antibody.

The term “capture antibody” as used herein means an antibody, whether polyclonal, monoclonal, or an immunoreactive fragment thereof, which is capable of binding an antigen of interest and thereby permit the recognition of the antigen by a subsequently applied antibody. The capture antibody can be used in either a heterogeneous (solid phase) or homogeneous (solution phase) assay. Preferably, the capture antibody is immobilized onto a solid phase.

The term “detection antibody” as used herein includes an antibody comprising a detectable label that is specific for (i.e., binds, is bound by, or forms a complex with) one or more analytes of interest in a sample. The term also encompasses an antibody that is specific for one or more analytes of interest, wherein the antibody can be bound by another species that comprises a detectable label. Examples of detectable labels include, but are not limited to, biotin/streptavidin labels, nucleic acid (e.g., oligonucleotide) labels, chemically reactive labels, fluorescent labels, enzyme labels, radioactive labels, and combinations thereof.

Antibodies can be fragmented using conventional techniques. Thus, the term “fragment thereof” includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, dabs and single chain antibodies (scFv) containing a VL and VH domain joined by a peptide linker. The scFv's may be covalently or non-covalently linked to form antibodies having two or more binding sites.

The term “antibody-based binding moiety” includes polyclonal, monoclonal, or other purified preparations of antibodies and recombinant antibodies. The term “antibody-based binding moiety” is further intended to include humanized antibodies, bi-specific antibodies, and chimeric molecules having at least one antigen-binding determinant derived from an antibody molecule.

In a preferred embodiment, the antibody-based binding moiety is detectably labeled, As used herein, “Labeled antibody” includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the methods of the invention that use antibody-based binding moieties for the detection of IL-8, the IL-8 level in a urine sample correlates to the intensity of the signal emitted from the detectably labeled antibody.

In one preferred embodiment, the antibody-based binding moiety is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydmgenase, alpha-glycerophosphate dehydrogenase, triose phosphate isomerase, horseradish peroxidase, alkaline phosphatase, asparaginase, glucose oxidase, β-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays, The radioactive isotope can be detected by means such as the use of a gamma counter or a scintillation counter or by audoradiography. Isotopes which are particularly useful for the purpose of the present invention are ³H, ³¹P, ³⁵S, ¹⁴C, and ¹²⁵I.

It is also possible to label an antibody with a fluorescent compound. When the fluorescently labeled antibody is exposed to light of the proper wavelength, its presence can be detected due to fluorescence. Among the most commonly used fluorescent labeling compounds are CYE dyes, fluorescein isothiocyanate, rhodamine, phycoerythrin, phycocyanin, allophycocyanin, o-phthaldehyde and fluorescamine. An antibody can also be detectably labeled using fluorescence emitting metals such as ¹⁵²Eu, or others of the lanthanide series. These metals can be attached to the antibody using metal-chelating groups such as diethylenetriaminepentaacetic acid (DTPA) or ethytenediaminetetraacetic acid (EDTA). An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

According to this invention, the term “a predetermined standard” may represent a normal range, a normal value, or a normal cutoff value of the creatinine-normalized level of IL-8 for healthy subjects as determined by a selected means. An appropriate cutoff value of the IL-8 level between normal individuals and patients with urolithiasis can be easily determined by those skilled in the art. Preferably, the normal individuals have normal urinalysis, normal physical examination and no known urolithiasis history, as well as urinary C-reactive protein (CRP) levels not greater than 0.1 mg/L. In addition, the creatinine-normalized level of IL-8 of a human subject as detected in a previous examination may also be used as the predetermined standard for the human subject.

In a preferred embodiment of this invention, the predetermined standard of the IL-8 level is measured by multiplex immunoassay using the BD™ Cytometric Bead Array (BD Biosciences, San Diego, Calif.), giving a value of 6.2 pg/mg creatinine as the creatinine-normalized cutoff value of the IL-8 level. As an alternative, the predetermined standard of the IL-8 level may be an average of the IL-8 levels (creatinine-normalized) of normal individuals and expressed as mean±2 standard deviations (SDs).

### EXAMPLES

The present invention will be described in more detail with reference to the following examples, which are given for the purpose of illustration only and are not intended to limit the scope of the present invention.

### A. Materials and Experimental Procedures:

### Study Subjects

Seventy individuals were recruited in the study of this invention using a protocol approved by the Institutional Review Board of Kaohsiung Medical University. In addition, informed consent was obtained from each of the human subjects studied in this invention for the donation of their urine samples. Patients were enrolled from the Department of Urology and controls from the Health Examination Center at the Kaohsiung Medical University Hospital. The patients recruited in the study of this invention required radiographic and ecographic documentation of urinary stones at the time of urine collection. Exclusion criteria included infection, hyperparathyroidism, hyperuricemia and presence of non-calcium renal stones. The controls had normal urinalysis, normal physical examination and no known urolithiasis history. To exclude subjects with subclinical infectious urinary inflammation, patients and controls whose urinary C-reactive protein (CRP) levels were greater than 0.1 mg/L were excluded from the study of this invention.

The clinical characteristics of patients with urolithiasis are shown in Table 1.

TABLE 1
Clinical information for control subjects and urolithiasis
patients examined in the study of this invention

| | Normal control | Urolithiasis |
|---|---|---|
| Number of cases | 38 | 32 |
| Age^{a}(range) (year) | 48 ± 16 (18-74) | 55 ± 15 (26-80) |
| Sex (female/male) | 11/27 | 8/24 |
| single/mutliple stones | NA^{b} | 19/13 |
| unilateral/bilateral | NA | 23/9 |
| Stone in kidney (%) | NA | 38 |
| Stone in ureter (%) | NA | 75 |
| Stone in bladder (%) | NA | 9 |
| Recurrent (%) | NA | 28 |

^{a}mean ± standard deviation
^{b}NA, not applicable

### Urine Collection and Biomarker Determination

Midstream morning urine from each of healthy volunteers and patients was kept at 4° C. and centrifuged at 1,000 g for 10 min. The resultant supernatant was divided into aliquots and stored at −70° C.

In the study of this invention, inflammatory cytokines and chemokines were detected by multiplex immunoassay. Concentrations of inflammatory cytokines and chemokines were measured by the BD™ Cytometric Bead Array (BD Biosciences, San Diego, Calif.), which contains microparticles that are dyed to exhibit different fluorescence intensities at approximately 650 nm. Each particle (capture bead) is coated with monoclonal antibodies against one of the cytokines (IL-1β, IL-6, IL-10, TNF-α, or IL-12p70) or chemokines (IL-8, RANTES, Mig, MCP-1 or IP-10) examined in the study of this invention. The cytokines and chemokines, after being bound to their respective capture beads, were directly detected using a mixture of corresponding antibodies conjugated with phycoerythrin (PE), which emits at 585 nm.

In the experiment, a mixture of capture beads was incubated with either standards (recombinant cytokines and chemokines) or test samples, followed by PE-conjugated detection antibodies to form sandwich complexes, according to the manufacturer's instructions. Thereafter, the reaction mixture was run on a FACSarray flow cytometer and analyzed using the Becton Dickinson Cytometric Bead Array software. The concentration of a recombinant cytokine or chemokine was proportional to its corresponding median fluorescence intensity detected at 585 nm.

The concentration of a cytokine or chemokine in a test sample was determined based on the standard curve of its corresponding recombinant cytokine or chemokine. The minimum thresholds for detection of each protein as examined in the study of this invention are as follows: IL-1β: 3.6 pg/mL; IL-6: 2.5 pg/mL; IL-10: 3.3 pg/mL; TNF-α: 3.7 pg/mL; IL-12p70; 2 pg/mL; IL-8: 0.2 pg/mL: RANTES: 1 pg/mL; Mig: 2.5 pg/mL; MCP-1: 3 pg/mL; and IP-10: 3 pg/mL.

### Statistics

To reduce dilution effects, the concentrations of cytokines and chemokines for each subject were normalized by creatinine levels and then expressed as mean±SD (pg/mg creatinine). In addition, the distribution of each biomarker in patients and controls was examined separately. Age and gender effects on these biomarkers were also evaluated. The area under the receiver operating characteristics (ROC) curve for each biomarker was calculated and used as an index to determine which biomarker had a greater diagnostic power to distinguish patients from controls.

The applicants also dichotomized biomarker levels to evaluate the differences of their levels between patients and controls by the chi-square test. The cutoff value to dichotomize each cytokine and chemokine was the mean plus 2 SDs calculated from controls. Significance was defined as a two-side p value<0.05.

### B. Results:

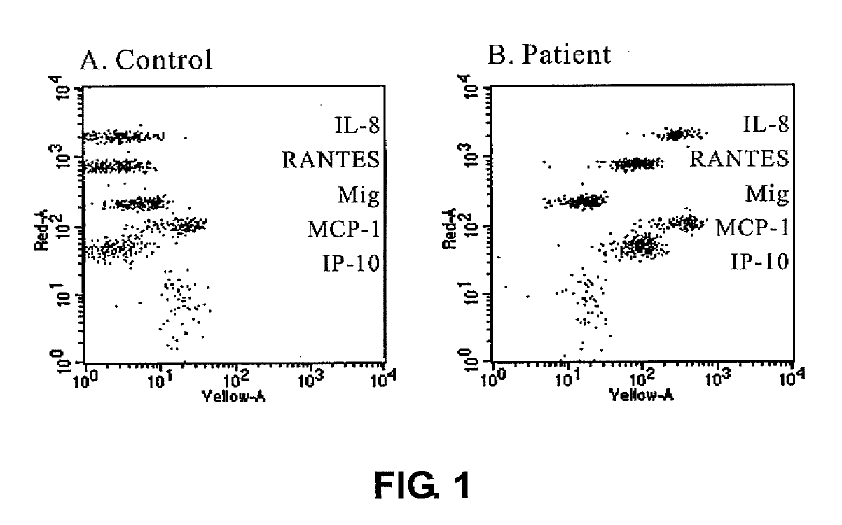

Representative data of chemokine levels using cytometric bead arrays are shown in FIG. 1. One outlier was found in the control group who had an IL-8/CXCL8 level greater than 5 group-specific SDs and, therefore, his data on IL-8/CXCL8 (275.46 pg/mg creatinine) was not analyzed.

The range of the creatinine-normalized value of each biomarker is shown in Table 2. The mean levels of IL-12p70, IL-10 and TNF-α were below the detectable values in both patients and controls. IL-6 levels were below the detectable threshold in controls. The ROC area was significant for the creatinine-normalized levels of IL-8 (area=0.84, p<0.001), Mig (area=0.76, p<0.001), and RANTE (area=0.66, p=0.024).

TABLE 2
The mean, standard deviation and range of each of the cytokines and
chemokines examined in the study of this invention

| | Urolithiasis | Control |
|---|---|---|
| | Mean^{b} ± SD (range) | Mean^{b} ± SD (range) |
| IL-8/CXCL8^{a} | 131.94 ± 242.04 (0-1157.97) | 7.97 ± 8.93 (0-33.82) |
| RANTES/CCL5 | 17.59 ± 30.19 (0-144.78) | 4.69 ± 3.06 (0-14.04) |
| Mlg/CXCL9 | 239.30 ± 218.81 (0-995.59) | 177.57 ± 149.03 (0-665.19) |
| MCP-1/CCL2 | 739.16 ± 1021.02 (0-3504.28) | 146.73 ± 95.66 (30.35-500.83) |
| IP-10/CXCL10 | 681.05 ± 765.10 (0-3236.31) | 257.09 ± 244.09 (0-904.06) |
| IL-1β | 11.11 ± 19.54 (0-79.17) | 4.59 ± 10.43 (0-44.15) |
| IL-6 | 13.26 ± 22.34 (0-91.72) | 1.61 ± 2.25 (0-7.22) |
| IL-10 | Below the detectable level | Below the detectable level |
| TNF-α | Below the detectable level | Below the detectable level |
| IL-12p70 | Below the detectable level | Below the detectable level |

^{a}The outlier was removed.
^{b}pg/mg creatinine.

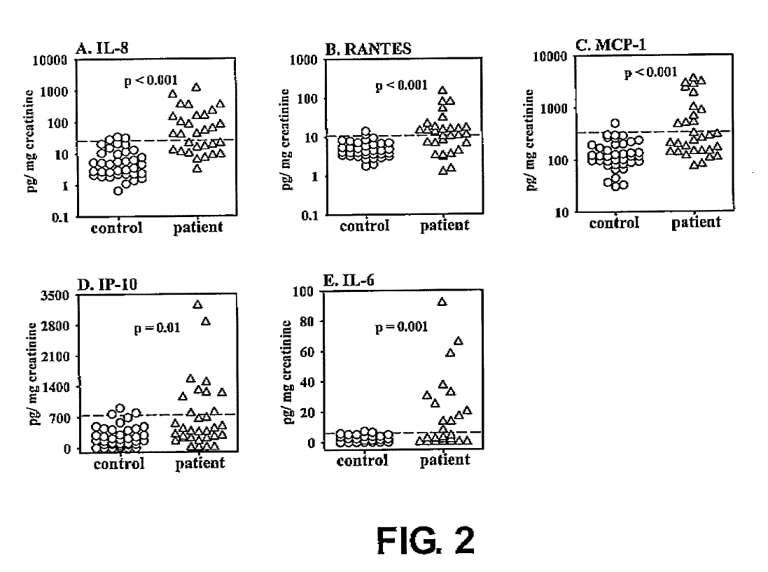

Based on the ROC curve, the cutoff point for the creatinine-normalized level of IL-8 was 6.2 pg/mg creatinine. As such, creatinine-normalized IL-8 levels≧6.2 pg/mg creatinine would indicate the existence of urinary stone. According to this cutoff point of IL-8, the diagnostic sensitivity and specificity can reach 91% and 68%, respectively. The other approach was to dichotomize every biomarker using an arbitrary cutoff point of mean plus 2 SDs. Using the chi-square test, IL-8, RANTES, MCP-1, IP-10 and IL-6 were significantly elevated in patients as compared to controls (FIG. 2).

From further analyses that compared patients with and without a previous history of urinary stone, the applicants did not find that recurrent cases had higher biomarker levels than first-attack patients. In addition, the biomarker levels were independent of single or multiple stones.

### C. Discussion:

The applicants' study reveals that the urinary concentrations of cytokines and chemokines in patients with urolithiasis are significantly higher than those in normal subjects. The significant biomarkers include IL-8, RANTES, ILL6, MCP-1, Mig and IP-10, amongst which IL8 is the most sensitive marker to distinguish patients from controls. The obtained data suggest a link between urinary stones and inflammation in the urinary tract. Although it is unclear whether inflammation contributes to urinary stone formation or vice versa, the applicants' findings may still provide a tool to detect high risk individuals.

Signs of inflammation have been associated with the presence of stone in the urinary tract. Evidence indicates that inflammation due to urolithiasis is different from inflammation caused by infectious agents.

Studies by Rhee and colleagues demonstrated that urinary levels of inflammatory cytokines such as IL-1β, IL-1α and IL-6 were markedly elevated in bacterial infection; however, only the JL-6 level was increased in urolithiasis (Rhee et al. (1998), supra). The applicants had a similar finding in the study of this invention, in which patients with infection based on clinical evidence or urinary levels of CRP greater than 0.1 mg/L were excluded. The applicants' results showed that several inflammatory chemokines and IL-6 in the urines of patients with urolithiasis were significantly higher as compared to the control subjects, but the levels of IL-1β were not different between the patients and the normal controls. This finding implies that urolithiasis may be associated with chronic subclinical non-infectious inflammation. The applicants' data show that the levels of TNF-α, IL-10 and IL-12p70 were undetectable in both patients and normal controls. This finding could be due to no increased production of these biomarkers in urolithiasis. However, it is also possible that these cytokines act mainly as local inflammatory mediators, or they are too diluted to be detectable in urine.

As for chemokines monitored in the study of this invention, they belong to the subset of inducible chemokines, which are up-regulated during inflammatory responses (Gerd Müller et al. (002), Journal of Leukocyte Biology, 72:1-8). The major function of inducible chemokines is to selectively control the chemotaxis of leukocytes to the inflamed tissues. Thus, the urinary levels of most inducible chemokines are higher than those of inflammatory cytokines even in normal individuals, as shown in Table 2. Accordingly, although the applicants' study did not demonstrate increased levels of TNF-α, IL-10 and IL-12p70, they may still be involved in the non-infectious inflammation of urolithiasis.

Due to the complexity and diversity of cells within the kidney, it is difficult to examine which cells play a major role in the recruitment of immune cells to the hyperoxaluric kidney in vivo. On the other hand, in vitro cell culture studies have provided important information. Previous studies have shown that tubular epithelial cells are a rich source of inducible chemokines including IL-8, RANTES and MCP-1 (Tohru Umekawa et al. (2002), supra; Stephan Segerer et al. (2000), J. Am. Soc. Nephrol., 11:152-176). Renal stones may contain appreciable amounts of endotoxin, and can stimulate epithelial cells to secrete chemokines via TLR4 (I. McAleer et al. (2003), Journal of Urology, 169(5):1813-1814). In fact, renal tubular cells can express several types of TLRs, including TLR1, 2, 3, 4 and 6, but not TLR5 and 9. Induction of RANTES and MCP-1 by renal tubular cells have been demonstrated after stimulation by TLR4 and TLR2 (Naotake Tsuboi et al. (2002), J. Immunol., 169:2026-2033). However, the in vivo role of tubular epithelial cells and renal tubular cells in chemokine production should be further examined.

The applicants' data demonstrate a significant increase of IL-8, RANTES, Mig, MCP-1, IP-10 and IL-6 urinary levels in patients with urolithiasis as compared to control subjects. These chemokines and cytokines can be secreted from tubular epithelial cells and renal tubular cells, as well as particular immune cell subsets. The applicants speculate that dendritic cells, NK cells, monocytes, T cells and neutrophils may be involved in the non-infectious inflammation of urolithiasis. For example, RANTES is a chemoattractant for immature dendritic cells and NK cells, Immature dendritic cells can also produce RANTES, IP-10, IL-8, MIP-1α and MIP-1β in response to agonists of TLR2 and TLR4 (Fabio Re and Jack L. Strominger (2001), supra). In addition, IFN-γ secreted by NK cells can also induce the production of IP-10 and Mig from resident tissue cells. IP-10 and Mig will then guide activated T cells back into the inflamed tissues (Thais P. Salazar-Mather et al. (2000), supra). The early production of these chemokines from tubular epithelial cells and dendritic cells is essential in shaping the immune response in kidney. Early expression of IP-10 induced by endogenous danger signals, such as trauma or hypoxia, was found to be important for the influx of T and NK cells (Wayne W. Hancock et al. (2001), J. Exp. Med., 193:975-980). IL-8 is known for neutrophil recruitment to an inflammatory site. MCP-1 not only recruits monocytes but also memory T cells and NK cells to mediate proinflammatory effects (Christine Daly and Barrett J. Rollins (2003), Microcirculation, 10:247-257). MCP-1 also can stimulate tubular epithelial cells to secrete IL-6 and express intercellular adhesion molecule-1 (Christiane Viedt et al. (2002), J Am Soc Nephrol., 13:1534-1547). Thus renal damage induced by stones can initiate a “chemokine to cytokine to chemokine” cascade, which may play a significant role in the disease process of urolithiasis.

The study of this invention shows that urinary IL-8 is a potential biomarker for the detection of urolithiasis. Although the present study cannot tell whether the elevated level of IL-8 is the cause or the result of urolithiasis, it still has the potential to become a screening biomarker. A follow-up study on high risk individuals may provide a better insight into the role of IL-8 in the development of urolithiasis.

All documents cited in the present specification as well as the references described therein, are hereby incorporated by reference in their entirety. In case of conflict, the present description, including definitions, will prevail.

While this invention has been described in connection with specific embodiments thereof, it will be understood that it is capable of further modifications and this application is intended to cover any variations, uses, or adaptations of the invention following, in general, the principles of the invention and including such departures from the present customary practice within the art to which the invention pertains and as may be applied to the essential features hereinbefore set forth, and as follows in the scope of the appended claims.

We claim:

1. A method for the detection or preliminary screening of urolithiasis in a human subject, comprising:
detecting the IL-8 level and the creatinine level in a urine sample taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
wherein an elevation of the creatinine-normalized IL-8 level in the urine sample as compared to the predetermined standard is indicative of urolithiasis.

2. The method according to claim 1, wherein the IL-8 level is detected by quantifying IL-8 using any one of the following methodologies: multiplex immunoassay, enzyme linked immunosorbent assay, radioimmunoassay, and immunoradiometric assay.

3. The method according to claim 1, wherein quantifying IL-8 is conducted using an antibody-based binding moiety which specifically binds IL-8.

4. The method according to claim 3, wherein the antibody-based binding moiety is an antibody.

5. The method according to claim 3, wherein the antibody-based binding moiety is labeled with a detectable label.

6. The method according to claim 5, wherein the label is selected from the group consisting of a radioactive label, a hapten label, a fluorescent label, and an enzymatic label.

7. The method according to claim 1, wherein detecting the IL-8 level is conducted by multiplex immunoassay, and the creatinine-normalized cutoff value of IL-8 is determined to be 6.2 pg/mg creatinine,

8. A method for monitoring urolithiasis in a human subject, comprising:
detecting the IL-8 level and the creatinine level in a urine sample periodically taken from a human subject suspected to have urolithiasis;
obtaining a creatinine-normalized IL-8 level in the urine sample by normalizing the detected IL-8 level to the detected creatinine level; and
comparing the creatinine-normalized IL-8 level in the urine sample with a predetermined standard;
wherein an elevation of the creatinine-normalized IL-8 level in the urine sample as compared to the predetermined standard is indicative of urolithiasis.

9. The method according to claim 8, wherein the IL-8 level is detected by quantifying IL-8 using any one of the following methodologies: multiplex immunoassay, enzyme linked immunosorbent assay, radioimmunoassay, and immunoradiometric assay.

10. The method according to claim 8, wherein quantifying IL-8 is conducted using an antibody-based binding moiety which specifically binds IL-8.

11. The method according to claim 9, wherein the antibody-based binding moiety is an antibody.

12. The method according to claim 9, wherein the antibody-based binding moiety is labeled with a detectable label.

13. The method according to claim 12, wherein the label is selected from the group consisting of a radioactive label, a hapten label, a fluorescent label, and an enzymatic label.

14. The method according to claim 8, wherein detecting the IL-8 level is conducted by multiplex immunoassay, and the creatinine-normalized cutoff value of IL-8 is determined to be 6.2 pg/mg creatinine.
